(19) United States
(12) Patent Application Publication
(10) Pub. No.: US 2010/0159515 A1
(43) Pub. Date: Jun. 24, 2010

(54) OXIDATIVE PRETREATMENT OF BIOMASS TO ENHANCE ENZYMATIC SACCHARIFICATION

(75) Inventor: Jelena Cirakovic, Wilmington, DE (US)

Correspondence Address: E I DU PONT DE NEMOURS AND COMPANY;LEGAL PATENT RECORDS CENTER, BARLEY MILL PLAZA 25/1122B, 4417 LANCASTER PIKE, WILMINGTON, DE, 19805, US

(73) Assignee: E.I. DU PONT DE NEMOURS AND COMPANY

(21) Appl. No.: 12/339,510

(22) Filed: Dec. 19, 2008

(51) Int. Cl. C12P 19/00; D21C 3/20; C12P 7/06; C12P 7/18; C12P 7/16; D21C 9/02

(52) U.S. Cl. 435/72; 162/79; 162/60; 162/77; 435/161; 435/160; 435/158

(57) ABSTRACT

Lignocellulosic biomass comprising lignin is treated by selective extraction and oxidation of lignin using a solvent solution comprising water in combination with at least one Mn(III) salt to produce readily saccharifiable carbohydrate enriched biomass.

### FIELD OF THE INVENTION

Methods for producing readily saccharifiable, carbohydrate-enriched lignocellulosic biomass are provided and disclosed. Specifically, pretreated biomass is prepared through simultaneous oxidative degradation and selective extraction of lignin under organosolv conditions at elevated temperatures in the presence of at least one manganese(III) oxidation catalyst. The remaining carbohydrate-enriched solids in the pretreated biomass may then be subjected to enzymatic saccharification to obtain fermentable sugars, which may be subjected to further processing for the production of other target products.

### BACKGROUND OF THE INVENTION

Cellulosic and lignocellulosic feedstocks and wastes, such as agricultural residues, wood, forestry wastes, sludge from paper manufacture, and municipal and industrial solid wastes, provide a potentially large renewable feedstock for the production of chemicals, plastics, fuels and feeds. Cellulosic and lignocellulosic feedstocks and wastes, composed of carbohydrate polymers comprising cellulose, hemicellulose, pectins and lignin are generally treated by a variety of chemical, mechanical and enzymatic means to release primarily hexose and pentose sugars, which can then be fermented to useful products.

Pretreatment methods are usually used to make the polysaccharides of lignocellulosic biomass more readily accessible to cellulolytic enzymes. One of the major impediments to cellulolytic enzyme digest of polysaccharide is the presence of lignin, a barrier that limits the access of the enzymes to their substrates, and a surface to which the enzymes bind non-productively. Because of the significant cost of enzyme in the pretreatment process, it is desirable to minimize the enzyme loading by either inactivation of the lignin to enzyme adsorption or its outright extraction. Another challenge is the inaccessibility of the cellulose to enzymatic hydrolysis either because of its protection by hemicellulose and lignin or by its crystallinity. Pretreatment methods that attempt to overcome these challenges include: steam explosion, hot water, dilute acid, ammonia fiber explosion, alkaline hydrolysis (including ammonia recycled percolation), oxidative delignification and organosolv.

While generally successful in lignin removal, organosolv methods as previously practiced for the treatment of lignocellulose biomass for either the production of pulp or for biofuels applications have suffered from poor sugar recoveries, particularly those of xylose. For example, the use of slightly acidic ethanol-water mixtures (e.g., EtOH 42 wt %) at elevated temperature to remove lignin from lignocellulosic biomass (Kleinert, T. N., Tappi 57: 99-102, 1974) resulted in substantial loss of carbohydrate. Dilute acid hydrolysis at 95° C. followed by organic solvent extraction and enzymatic saccharification (Lee, Y-H. et al., Biotech. Bioeng., 29: 572-581, 1987) resulted in substantial loss of hemicellulose during hydrolysis, additional carbohydrate loss upon organic solvent extraction and poor yield (˜50% of total carbohydrate) upon enzymatic saccharification of residue.

Additional shortcomings of previously applied methods include, separate hexose and pentose streams (e.g. dilute acid), inadequate lignin extraction or lack of separation of extracted lignin from polysaccharide, particularly in those feedstocks with high lignin content (e.g., sugar cane bagasse, softwoods), disposal of waste products (e.g., salts formed upon neutralization of acid or base), and poor recoveries of carbohydrate due to breakdown or loss in wash steps. Other problems include the high cost of energy, capital equipment, and pretreatment catalyst recovery, and incompatibility with saccharification enzymes.

A number of pretreatment methods involving the use of manganese to remove lignin from biomass have been disclosed. For example, U.S. Pat. No. 3,939,286 discloses a process for treating plant organic matter to increase the digestability thereof by ruminants. The process includes mixing the organic particles with water, a nontoxic acid catalyst to produce a pH lower than 3.0, and a metallic catalyst of either iron or manganese, oxidizing the mixture under elevated pressure and temperature, and hydrolyzing the oxidized mixture under elevated pressure and temperature to convert at least a portion of the cellulose molecules to saccharides and saccharide acids.

U.S. Pat. No. 4,087,318 discloses a process for the delignification of lignocellulosic material wherein the lignocellulosic material, prior to the delignification, is treated with water or an aqueous solution to remove compounds which catalyze the degradation of carbohydrates and then the delignification is carried out with oxygen and alkali in the presence of a manganese compound to improve the selectivity of the delignification and increase the rate of delignification.

U.S. Pat. No. 5,630,906 discloses a method for delignifying and bleaching a lignocellulose material, wherein an aqueous solution of a redox catalyst and an oxidant is reacted with the material. The catalyst comprises an organometallic cation of the general formula [(L)MnO₂Mn(L)]ⁿ⁺, wherein Mn is manganese(III) or (IV) oxide, the two Mn's of this cation may form a pair in a III-III, III-IV, or IV-IV oxidative state, n is 2, 3, or 4, O is oxygen, and L is a ligand comprising four nitrogen atoms coordinating the manganese.

Published patent application WO 01/77031 discloses a process for removing phenols from an aqueous solution. The process uses a metal oxide, such as titanium dioxide, for the selective adsorption and removal of phenolic compounds from an aqueous solution, such as a biomass-hydrolyzate medium. The use of manganese dioxide is claimed.

U.S. Pat. No. 6,770,168 discloses a substantially sulfur free process for the manufacturing of a chemical pulp with an integrated recovery system for recovery of pulping chemicals. The process is carried out in several stages involving physical and chemical treatment of lignocellulosic material in order to increase accessibility of the lignocellulosic material to reactions with an oxygen-based delignification agent. Following the chemical and physical pretreatment the material is reacted with an oxygen-containing gas in the presence of an alkaline buffer solution and in the presence of one or more active chemical reagents in order to obtain a delignified brown stock pulp. The preferred oxygen delignification catalysts comprise at least one of the metals copper, manganese, iron, cobalt, or ruthenium.

One of the major challenges of the pretreatment of lignocellulosic biomass is to maximize the extraction or chemical neutralization (with respect to non-productive binding of cellulolytic enzymes) of the lignin while minimizing the loss of carbohydrate (cellulose plus hemicellulose). The higher the selectivity, the higher the overall yield of monomeric sugars following combined pretreatment and enzymatic saccharification.

In this disclosure, a combination of manganese-mediated fragmentation and selective organosolv extraction of lignin at elevated temperatures is used to produce carbohydrate-enriched biomass in a cost effective process. The carbohydrate-enriched biomass is highly susceptible to enzymatic saccharification, producing high yields of fermentable sugars (for example, glucose and xylose) for their bioconversion to value-added chemicals and fuels.

### SUMMARY OF THE INVENTION

The present invention provides a method for producing readily saccharifiable carbohydrate-enriched biomass and for selectively extracting lignin from lignocellulosic biomass while retaining carbohydrate in good yield. The methods include treating lignocellulosic biomass with a solvent solution, such as organosolv, in the presence of at least one Mn(III) salt at elevated temperatures. Following pretreatment, the biomass may be further treated with a saccharification enzyme consortium to produce fermentable sugars. These sugars may be subjected to further processing for the production of target products. In one embodiment of the invention, a method is provided, the method comprising:

(a) providing lignocellulosic biomass comprising lignin;

(b) contacting the biomass with a solvent solution comprising water in the presence of at least one Mn(III) salt whereby a biomass-solvent suspension is formed;

(c) heating the biomass-solvent suspension to a temperature of about 100° C. to about 220° C. for a reaction time of about 15 minutes to about 48 hours whereby lignin is fragmented from the biomass and said lignin is dissolved in the suspension; and

(d) filtering an amount of free liquid under pressure after heating the suspension in (c) whereby the dissolved lignin is removed and whereby readily saccharifiable biomass is produced.

In another embodiment, a method for selectively removing lignin from biomass is provided, the method comprising:

(a) providing lignocellulosic biomass having a carbohydrate content and comprising lignin;

(b) contacting the biomass with a solvent solution comprising water in the presence of at least one Mn(III) salt whereby a biomass-solvent suspension is formed;

(c) heating the biomass-solvent suspension to a temperature of about 100° C. to about 220° C. for a reaction time of about 15 minutes to about 48 hours whereby lignin is fragmented from the biomass and is dissolved in the suspension; and

(d) filtering free liquid under pressure after heating the suspension in (c) whereby dissolved lignin is removed and wherein the carbohydrate content of the biomass is highly conserved.

According to the methods of the invention, the Mn(III) salt is selected from the group consisting of manganese(III) triacetate, manganese(III) acetylacetonate, and combinations thereof. According to the methods of the invention, the Mn(III) salt is at a concentration of up to about 15% by weight of dry biomass. In some embodiments, the Mn(III) salt concentration is up to about 10%. In some embodiments, the Mn(III) salt concentration is up to about 5%. The Mn(III) salt may be formed in situ during the pretreatment processes described herein.

According to the methods of the invention, the solvent comprises an alcohol selected from the group consisting of methanol, ethanol, n-propanol, isopropanol, n-butanol, 2-butanol, isobutanol, and t-butanol, and mixtures of these. In some embodiments, the alcohol is ethanol. In some embodiments, the solvent solution contains about 0 percent to about 100 percent (v/v) ethanol. In some embodiments, the ethanol/water treatment solution contains about 10 percent to about 90 percent (v/v) ethanol. In some embodiments, the ethanol/water treatment solution contains about 25 percent to about 75 percent (v/v) ethanol.

According to the methods of the invention, the dry weight of biomass is at a concentration of from about 15% to about 70% of the weight of the biomass suspension. In some embodiments, the dry weight of biomass is from about 20% to about 50% of the weight of the biomass suspension. In some embodiments, the method further comprises drying the filtered biomass after step (d).

The methods described herein may be repeated to achieve maximal results.

### DETAILED DESCRIPTION OF THE INVENTION

Applicants specifically incorporate the entire contents of all cited references in this disclosure. Further, when an amount, concentration, or other value or parameter is given as either a range, preferred range, or a list of upper preferable values and lower preferable values, this is to be understood as specifically disclosing all ranges formed from any pair of any upper range limit or preferred value and any lower range limit or preferred value, regardless of whether ranges are separately disclosed. Where a range of numerical values is recited herein, unless otherwise stated, the range is intended to include the endpoints thereof, and all integers and fractions within the range. It is not intended that the scope of the invention be limited to the specific values recited when defining a range.

The present invention provides a process for the treatment of biomass in order to enhance the subsequent enzymatic saccharification step. A process involving a pretreatment step wherein lignin is simultaneously fragmented, oxidized, and extracted using organosolv conditions at elevated temperatures in the presence of at least one Mn(III) salt is employed. The treated biomass is then filtered and washed to remove solubilized lignin, acetic acid, acetamides, and manganese salts. After filtering, the treated biomass may be dried. The biomass may be digested with a saccharification enzyme consortium to produce fermentable sugars.

### DEFINITIONS

The following definitions are used in this disclosure:

“Room temperature” and “ambient” when used in reference to temperature refer to any temperature from about 15° C. to about 25° C.

“Fermentable sugars” refers to a sugar content primarily comprising monosaccharides and some polysaccharides that can be used as a carbon source by a microorganism in a fermentation process to produce a target product.

“Lignocellulosic” refers to material comprising both lignin and cellulose. Lignocellulosic material may also comprise hemicellulose. In the methods described herein, lignin is dissolved and substantially removed from the lignocellulosic biomass to produce a carbohydrate-enriched biomass comprising fermentable sugars.

“Dissolved lignin” means the lignin that is dissolved in a solvent.

“Al lignin” refers to acid-insoluble lignin.

“Autohydrolysis” refers to the hydrolysis of biomass in the presence of solvent (water or organic solvent plus water) plus heat with no further additions, such as without hydrolytic enzymes

“Cellulosic” refers to a composition comprising cellulose.

“Target product” refers to a chemical, fuel, or chemical building block produced by fermentation. Product is used in a broad sense and includes molecules such as proteins, including, for example, peptides, enzymes, and antibodies. Also contemplated within the definition of target product are ethanol and butanol.

The abbreviation “EtOH” refers to ethanol or ethyl alcohol.

“Dry weight of biomass” refers to the weight of the biomass having all or essentially all water removed. Dry weight is typically measured according to American Society for Testing and Materials (ASTM) Standard E1756-01 (Standard Test Method for Determination of Total Solids in Biomass) or Technical Association of the Pulp and Paper Industry, Inc. (TAPPI) Standard T-412 om-02 (Moisture in Pulp, Paper and Paperboard).

“Selective extraction” means removal of lignin while substantially retaining carbohydrates.

A “solvent” or “solvent solution” as used herein is a liquid that dissolves a solid, liquid, or gaseous solute, resulting in a solution. The most suitable solvents for this invention include organic solvents such as methanol, ethanol, n-propanol, isopropanol, n-butanol, 2-butanol, isobutanol, and t-butanol. The solvent solutions as used herein also include organic solvent solutions that may be in a mixture with other components.

“Biomass” and “lignocellulosic biomass” as used herein refer to any lignocellulosic material, including cellulosic and hemi-cellulosic material, for example, bioenergy crops, agricultural residues, municipal solid waste, industrial solid waste, yard waste, wood, forestry waste, and combinations thereof, and as further described below. Biomass has a carbohydrate content that comprises polysaccharides and oligosaccharides and may also comprise additional components, such as protein and/or lipid.

“Highly conserved” as used herein refers to the carbohydrate content of the lignocellulosic material after the processing steps described herein. In an embodiment of the invention, the highly conserved carbohydrate content provides for sugar yields after saccharification that are substantially similar to theoretical yields and/or demonstration of minimal loss in sugar yield from the processes described herein. In an embodiment of the invention, highly-conserved with reference to carbohydrate content refers to the conservation of greater than or equal to 85% of the biomass carbohydrate as compared to biomass prior to pretreating as described herein.

“Preprocessing” as used herein refers to processing of lignocellulosic biomass prior to pretreatment. Preprocessing is any treatment of biomass that prepares the biomass for pretreatment, such as mechanically chopping and/or drying to the appropriate moisture contact.

“Biomass-solvent suspension” refers to a mixture of biomass and solvent wherein the biomass is in suspension in the solvent solution. The biomass suspension may comprise additional components such as at least one Mn(III) salt.

“Saccharification” refers to the production of fermentable sugars from polysaccharides by the action of hydrolytic enzymes. Production of fermentable sugars from pretreated biomass occurs by enzymatic saccharification by the action of cellulolytic and hemicellulolytic enzymes.

“Pretreating biomass” or “biomass pretreatment” as used herein refers to subjecting native or preprocessed biomass to chemical, physical, or biological action, or any combination thereof, rendering the biomass more susceptible to enzymatic saccharification or other means of hydrolysis prior to saccharification. For example, the methods claimed herein may be referred to as pretreatment processes that contribute to rendering biomass more accessible to hydrolytic enzymes for saccharification.

“Pretreated biomass” as used herein refers to native or preprocessed biomass that has been subjected to chemical, physical, or biological action, or any combination thereof, rendering the biomass more susceptible to enzymatic saccharification or other means of hydrolysis prior to saccharification.

“Air-drying the filtered biomass” can be performed by allowing the biomass to dry through equilibration with the air of the ambient atmosphere.

“Readily saccharifiable biomass” means biomass that is carbohydrate-enriched and made more amenable to hydrolysis by cellulolytic or hemi-cellulolytic enzymes for producing monomeric and oligomeric sugars.

“Carbohydrate-enriched” as used herein refers to the biomass produced by the process treatments described herein. In one embodiment the readily saccharifiable carbohydrate-enriched biomass produced by the processes described herein has a carbohydrate concentration of greater than or equal to about 85% of the biomass carbohydrate as compared to biomass prior to pretreating as described herein while removing 75% or greater of the biomass lignin.

“Heating the biomass suspension” means subjecting the biomass suspended in solvent to a temperature greater than ambient or room temperature. Temperatures relevant to the present pretreatments are from about 100° C. to about 220° C., or from about 140° C. to about 180° C., or any temperature within or approximately within these ranges.

“Filtering free liquid under pressure” means removal of unbound liquid through filtration, with some pressure difference on opposite faces of the filter.

“Air-dried sample” means a pretreated biomass which is allowed to dry at ambient temperature to the point where its moisture content is approximately in equilibrium with that of the ambient air, typically ≧85% dry matter.

“Substantially lignin-free biomass” means a pretreated sample containing about ≦25% of the starting lignin composition.

“Multi-component solvent” means a solvent containing organic solvent, water, and reagents capable of chemical attack on the lignin.

“Pressure vessel” is a sealed vessel that may be equipped or not with a mechanism for agitation of a biomass/solvent suspension, in which a positive pressure is developed upon heating the lignocellulosic biomass.

“Hydrolysate” refers to the liquid in contact with the lignocellulose biomass which contains the products of hydrolytic reactions acting upon the biomass (either enzymatic or not), in this case monomeric and oligomeric sugars.

“Organosolv” means a mixture of organic solvent and water.

“Solvent solution” as used herein refers to organic solvents in solution, such as an organosolv solution.

“Enzyme consortium” or “saccharification enzyme consortium” is a collection of enzymes, usually secreted by a microorganism, which in the present case will typically contain one or more cellulases, xylanases, glycosidases, ligninases and feruloyl esterases.

“Monomeric sugars” or “simple sugars” consist of a single pentose or hexose unit, e.g., glucose, xylose, and arabinose.

“Delignification” is the act of removing lignin from lignocellulosic biomass. In the context of this application delignification means fragmentation and extraction of lignin from the lignocellulosic biomass using organosolv at elevated temperatures in the in the presence of at least one Mn(III) salt.

“Simultaneous fragmentation” is a fragmentation reaction performed in organosolv solvent such that the fragments go into solution as soon as they are released from the bulk biomass.

“Fragmentation” is a process in which lignocellulosic biomass is treated under organosolv conditions in the presence of at least one Mn(III) salt to break the lignin down into smaller subunits. In the context of the present application, oxidation of the lignin may contribute to breaking the lignin down into smaller subunits.

“Selective extraction” is a process by which lignin is extracted and dissolved by treatment under organosolv conditions leaving behind the polysaccharide.

Methods for pretreating lignocellulosic biomass to produce readily saccharifiable biomass are provided. These methods provide economic processes for rendering components of the lignocellulosic biomass more accessible or more amenable to enzymatic saccharification. The pretreatment can be chemical, physical, or biological, or any combination of the foregoing. In this disclosure the pretreatment is performed in the presence of a Mn(III) salt which promotes oxidation of the lignin. The presence of organosolv assists lignin fragmentation and removal and carbohydrate recovery.

In addition, the methods described in the present disclosure minimize the loss of carbohydrate during the pretreatment process and maximize the yield of monomeric sugars in saccharification.

As discussed above the methods described herein include pretreating lignocellulosic material with a solvent solution comprising the components described below to produce a readily saccharifiable carbohydrate-enriched biomass.

### Solvents:

The methods described herein include use of a solvent for pretreating biomass. Solvent solutions useful in the present methods are frequently referred to in the art as Organosolv. Details on pretreatment technologies related to use of solvents and other pretreatments can be found, for example, in Wyman et al., (Bioresource Tech. 96:1959, 2005); Wyman et al., (Bioresource Tech. 96:2026, 2005); Hsu, (“Pretreatment of biomass” In Handbook on Bioethanol: Production and Utilization, Wyman, Taylor and Francis Eds., p. 179-212, 1996); and Mosier et al., (Bioresource Tech. 96:673, 2005). Solvents are used herein for pretreating biomass to remove lignin. Delignification is typically conducted at temperatures of 165-225° C., at liquid to biomass ratios of 4:1 to 20:1, at liquid compositions of 50% organic solvent to water (volume/volume [v/v]), and for reaction times between 0.5-12 hours. A number of mono- and polyhydroxy-alcohols have been tested as solvents. Ethanol, butanol and phenol have been used (Park, J. K., and Phillips, J. A., Chem. Eng. Comm., 65:187-205, 1988).

The organosolv or organic solvent pretreatment in the present methods may comprise a mixture of water and an organic solvent at selected condition parameters that include temperature, time, pressure, solvent-to-water ratio and solids-to-liquid ratio. The solvent can comprise, but is not limited to, alcohols, organic acids and ketones. The alcohols can be selected from the group consisting of methanol, ethanol, propanol, isopropanol, butanol, isobutanol, t-butyl alcohol, and mixtures of these. The alcohol can also be a glycol. The concentration of the solvent in solution (i.e. water) in the present invention is from about 0%-100% (v/v), or from about 10% to about 90%, or from about 25% to about 75%, or from about 40% to about 60% (v/v). Specifically, for purposes of an embodiment of the methods herein, EtOH/H₂O mixtures from about 0%-100% (v/v) ethanol were examined and solutions containing about 25-75% (v/v) EtOH were found to be most effective.

### Manganese(III) Salt:

According to the present method, the biomass is contacted with the solvent solution in the presence of at least one Mn(III) salt. Addition of a Mn(III) salt which can promote oxidation of the lignin is beneficial to pretreatment and results in an increased accessibility of the carbohydrate-enriched biomass to enzymatic saccharification. In the present invention, concentrations of Mn(III) salt up to about 15 weight percent (wt %) based on the weight of dry biomass can be used, for example up to about 10 wt %, or for example up to about 5 wt %. Higher concentrations can also be used and are effective but are generally not economical.

The Mn(III) salt can be added to the biomass suspension or it can be formed in situ, for example by oxidation of a Mn(II) salt or by reaction of a Mn(III) compound with appropriate ligands, such as acetate or acetylacetonate groups. Examples of Mn(III) species which are suitable for use in the current process include, but are not limited to, manganese(III) triacetate, manganese(III) acetylacetonate, and combinations thereof. The anion of the Mn(III) salt can be chosen from a variety of anions, or a mixture, as long as the anion is not detrimental to the process. The anion may be selected based on cost and availability of the Mn(III) salt, for example. As the Mn(III) promotes oxidation of the lignin, the metal is reduced. To be reused in the process, the active Mn(III) oxidation state would need to be regenerated.

### Lignocellulosic Biomass:

The lignocellulosic biomass pretreated herein includes, but is not limited to, bioenergy crops, agricultural residues, municipal solid waste, industrial solid waste, sludge from paper manufacture, yard waste, wood and forestry waste. Examples of biomass include, but are not limited to corn cobs, crop residues such as corn husks, corn stover, grasses, wheat, wheat straw, barley, barley straw, hay, rice straw, switchgrass, waste paper, sugar cane bagasse, sorghum, soy, components obtained from milling of grains, trees, branches, roots, leaves, wood chips, sawdust, shrubs and bushes, vegetables, fruits, flowers and animal manure.

In one embodiment, biomass that is useful for the invention includes biomass that has a relatively high carbohydrate content, is relatively dense, and/or is relatively easy to collect, transport, store and/or handle.

In one embodiment of the invention, biomass that is useful includes corn cobs, corn stover, sugar cane bagasse and switchgrass.

In another embodiment, the lignocellulosic biomass includes agricultural residues such as corn stover, wheat straw, barley straw, oat straw, rice straw, canola straw, and soybean stover; grasses such as switch grass, miscanthus, cord grass, and reed canary grass; fiber process residues such as corn fiber, beet pulp, pulp mill fines and rejects and sugar cane bagasse; sorghum; forestry wastes such as aspen wood, other hardwoods, softwood and sawdust; and post-consumer waste paper products; as well as other crops or sufficiently abundant lignocellulosic material.

The lignocellulosic biomass may be derived from a single source, or biomass can comprise a mixture derived from more than one source; for example, biomass could comprise a mixture of corn cobs and corn stover, or a mixture of stems or stalks and leaves.

In the present method, the biomass dry weight is at an initial concentration of at least about 10% up to about 80% of the weight of the biomass-solvent suspension during pretreatment. More suitably, the dry weight of biomass can be at a concentration of from about 15% to about 70%, or about 15% to about 60%, or about 20% to about 50% of the weight of the biomass-solvent suspension. The percent of biomass in the biomass-solvent suspension is kept high to reduce the total volume of pretreatment material, decreasing the amount of solvent and reagents required and making the process more economical.

The biomass may be used directly as obtained from the source, or may be subjected to some preprocessing, for example, energy may be applied to the biomass to reduce the size, increase the exposed surface area, and/or increase the accessibility of lignin and of cellulose, hemicellulose, and/or oligosaccharides present in the biomass to organosolv pretreatment and to saccharification enzymes used in the second step of the method. Energy means useful for reducing the size, increasing the exposed surface area, and/or increasing the accessibility of the lignin, and the cellulose, hemicellulose, and/or oligosaccharides present in the biomass to the organosolv pretreatment and to saccharification enzymes include, but are not limited to, milling, crushing, grinding, shredding, chopping, disc refining, ultrasound, and microwave. This application of energy may occur before or during pretreatment, before or during saccharification, or any combination thereof.

Drying biomass prior to pretreatment may occur as well by conventional means, such as by using rotary dryers, flash dryers, or superheated steam dryers.

### Pretreatment Conditions:

Pretreatment of biomass with the solvent solution in the presence of at least one Mn(III) salt is carried out in any suitable vessel. Typically the vessel is one that can withstand pressure, has a mechanism for heating, and has a mechanism for mixing the contents. Commercially available vessels include, for example, the Zipperclave® reactor (Autoclave Engineers, Erie, Pa.), the Jaygo reactor (Jaygo Manufacturing, Inc., Mahwah, N.J.), and a steam gun reactor ((described in General Methods Autoclave Engineers, Erie, Pa.). Much larger scale reactors with similar capabilities may be used. Alternatively, the biomass and organosolv solution may be combined in one vessel, then transferred to another reactor. Also biomass may be pretreated in one vessel, then further processed in another reactor such as a steam gun reactor (described in General Methods; Autoclave Engineers, Erie, Pa.).

The pretreatment reaction may be performed in any suitable vessel, such as a batch reactor or a continuous reactor. One skilled in the art will recognize that at higher temperatures (above 100° C.), a pressure vessel is required. The suitable vessel may be equipped with a means, such as impellers, for agitating the biomass-organosolv mixture. Reactor design is discussed in Lin, K.-H., and Van Ness, H. C. (in Perry, R. H. and Chilton, C. H. (eds), Chemical Engineer's Handbook, 5^{th} Edition (1973) Chapter 4, McGraw-Hill, NY). The pretreatment reaction may be carried out as a batch process, or as a continuous process.

Prior to contacting the biomass with solvent, vacuum may be applied to the vessel containing the biomass. By evacuating air from the pores of the biomass, better penetration of the solvent into the biomass may be achieved. The time period for applying vacuum and the amount of negative pressure that is applied to the biomass will depend on the type of biomass and can be determined empirically so as to achieve optimal pretreatment of the biomass (as measured by the production of fermentable sugars following saccharification).

The heating of the biomass with the solvent solution in the presence of at least one Mn(III) salt is carried out at a temperature of from about 100° C. to about 220° C. The heated solution may then be cooled rapidly to room temperature. In another embodiment, the heating of the biomass is carried out at a temperature of about 140° C. to about 180° C. In another embodiment, the heating of the biomass is carried out at a temperature of about 150° C. to about 170° C. Heating of the biomass-solvent suspension may occur for about 15 minutes to about 48 hours, or more preferably from about 1 hour to about 12 hours, or for example from about 1 hour to about 6 hours. In one embodiment, the contacting of the biomass is carried out at a temperature of about 150° C. for about 6 hours.

The contacting of the biomass with the solvent solution in the presence of at least one Mn(III) salt can be performed at autogeneous pressure. Higher or lower pressures can also be used but are generally less practical.

For the pretreatment process, the temperature, time for pretreatment, solvent solution, Mn(III) salt concentration, biomass concentration, biomass type, and biomass particle size are related; thus these variables may be adjusted as necessary for each type of biomass to optimize the pretreatment processes described herein.

To assess performance of the pretreatment. i.e., the production of readily saccharifiable biomass and subsequent saccharification, separately or together, the theoretical yield of sugars derivable from the starting biomass can be determined and compared to measured yields.

### Further Processing:

Saccharification:

Following pretreatment, the readily saccharifiable biomass comprises a mixture of organosolv solvent, Mn(III) and Mn(II) salts, oxidized, fragmented and extracted lignin, and polysaccharides. Prior to further processing, the manganese salts and lignin fragments or oxidation products may be removed from the pretreated biomass by filtration and washing the sample with EtOH/H₂O (0% to 100% EtOH v/v). The biomass may then be dried at room temperature resulting in readily saccharifiable biomass. The concentration of glucan, xylan and acid-insoluble lignin content of the readily saccharifiable biomass may be determined using analytical means well known in the art.

The readily saccharifiable biomass may then be further hydrolyzed in the presence of a saccharification enzyme consortium to release oligosaccharides and/or monosaccharides in a hydrolysate. Surfactants such as polyethylene glycols (PEG) may be added to improve the saccharification process (U.S. Pat. No. 7,354,743 B2, incorporated herein by reference). Saccharification enzymes and methods for biomass treatment are reviewed in Lynd, L. R., et al. (Microbiol. Mol. Biol. Rev., 66:506-577, 2002). The saccharification enzyme consortium may comprise one or more glycosidases; the glycosidases may be selected from the group consisting of cellulose-hydrolyzing glycosidases, hemicellulose-hydrolyzing glycosidases, and starch-hydrolyzing glycosidases. Other enzymes in the saccharification enzyme consortium may include peptidases, lipases, ligninases and feruloyl esterases.

The saccharification enzyme consortium comprises one or more enzymes selected primarily, but not exclusively, from the group “glycosidases” which hydrolyze the ether linkages of di-, oligo-, and polysaccharides and are found in the enzyme classification EC 3.2.1.x (Enzyme Nomenclature 1992, Academic Press, San Diego, Calif. with Supplement 1 (1993), Supplement 2 (1994), Supplement 3 (1995, Supplement 4 (1997) and Supplement 5 [in Eur. J. Biochem., 223:1-5, 1994; Eur. J. Biochem., 232:1-6, 1995; Eur. J. Biochem., 237:1-5, 1996; Eur. J. Biochem., 250:1-6, 1997; and Eur. J. Biochem., 264:610-650 1999, respectively]) of the general group “hydrolases” (EC 3.). Glycosidases useful in the present method can be categorized by the biomass component that they hydrolyze. Glycosidases useful for the present method include cellulose-hydrolyzing glycosidases (for example, cellulases, endoglucanases, exoglucanases, cellobiohydrolases, β-glucosidases), hemicellulose-hydrolyzing glycosidases (for example, xylanases, endoxylanases, exoxylanases, β-xylosidases, arabino-xylanases, mannases, galactases, pectinases, glucuronidases), and starch-hydrolyzing glycosidases (for example, amylases, α-amylases, β-amylases, glucoamylases, α-glucosidases, isoamylases). In addition, it may be useful to add other activities to the saccharification enzyme consortium such as peptidases (EC 3.4.x.y), lipases (EC 3.1.1.x and 3.1.4.x), ligninases (EC 1.11.1.x), and feruloyl esterases (EC 3.1.1.73) to help release polysaccharides from other components of the biomass. It is well known in the art that microorganisms that produce polysaccharide-hydrolyzing enzymes often exhibit an activity, such as cellulose degradation, that is catalyzed by several enzymes or a group of enzymes having different substrate specificities. Thus, a “cellulase” from a microorganism may comprise a group of enzymes, all of which may contribute to the cellulose-degrading activity. Commercial or non-commercial enzyme preparations, such as cellulase, may comprise numerous enzymes depending on the purification scheme utilized to obtain the enzyme. Thus, the saccharification enzyme consortium of the present method may comprise enzyme activity, such as “cellulase”, however it is recognized that this activity may be catalyzed by more than one enzyme.

Saccharification enzymes may be obtained commercially, in isolated form, such as Spezyme® CP cellulase (Genencor International, Rochester, N.Y.) and Multifect® xylanase (Genencor). In addition, saccharification enzymes may be expressed in host organisms at the biofuels plant, including using recombinant microorganisms.

One skilled in the art would know how to determine the effective amount of enzymes to use in the consortium and adjust conditions for optimal enzyme activity. One skilled in the art would also know how to optimize the classes of enzyme activities required within the consortium to obtain optimal saccharification of a given pretreatment product under the selected conditions.

Preferably the saccharification reaction is performed at or near the temperature and pH optima for the saccharification enzymes. The temperature optimum used with the saccharification enzyme consortium in the present method ranges from about 15° C. to about 100° C. In another embodiment, the temperature optimum ranges from about 20° C. to about 80° C. and most typically 45-50° C. The pH optimum can range from about 2 to about 11. In another embodiment, the pH optimum used with the saccharification enzyme consortium in the present method ranges from about 4 to about 5.5.

The saccharification can be performed for a time of about several minutes to about 120 hours, and preferably from about several minutes to about 48 hours. The time for the reaction will depend on enzyme concentration and specific activity, as well as the substrate used and the environmental conditions, such as temperature and pH. One skilled in the art can readily determine optimal conditions of temperature, pH and time to be used with a particular substrate and saccharification enzyme(s) consortium.

The saccharification can be performed batch-wise or as a continuous process. The saccharification can also be performed in one step, or in a number of steps. For example, different enzymes required for saccharification may exhibit different pH or temperature optima. A primary treatment can be performed with enzyme(s) at one temperature and pH, followed by secondary or tertiary (or more) treatments with different enzyme(s) at different temperatures and/or pH. In addition, treatment with different enzymes in sequential steps may be at the same pH and/or temperature, or different pHs and temperatures, such as using hemicellulases stable and more active at higher pHs and temperatures followed by cellulases that are active at lower pHs and temperatures.

The degree of solubilization of sugars from biomass following saccharification can be monitored by measuring the release of monosaccharides and oligosaccharides. Methods to measure monosaccharides and oligosaccharides are well known in the art. For example, the concentration of reducing sugars can be determined using the 1,3-dinitrosalicylic (DNS) acid assay (Miller, G. L., Anal. Chem., 31: 426-428, 1959). Alternatively, sugars can be measured by HPLC using an appropriate column as described below.

### Fermentation to Target Products:

The readily saccharifiable biomass produced by the present methods may be hydrolyzed by enzymes as described above to produce fermentable sugars which then can be fermented into a target product. “Fermentation” refers to any fermentation process or any process comprising a fermentation step. Target products include, without limitation alcohols (e.g., arabinitol, butanol, ethanol, glycerol, methanol, 1,3-propanediol, sorbitol, and xylitol); organic acids (e.g., acetic acid, acetonic acid, adipic acid, ascorbic acid, citric acid, 2,5-diketo-D-gluconic acid, formic acid, fumaric acid, glucaric acid, gluconic acid, glucuronic acid, glutaric acid, 3-hydroxypropionic acid, itaconic acid, lactic acid, malic acid, malonic acid, oxalic acid, propionic acid, succinic acid, and xylonic acid); ketones (e.g., acetone); amino acids (e.g., aspartic acid, glutamic acid, glycine, lysine, serine, and threonine); gases (e.g., methane, hydrogen (H₂), carbon dioxide (CO₂), and carbon monoxide (CO)).

Fermentation processes also include processes used in the consumable alcohol industry (e.g., beer and wine), dairy industry (e.g., fermented dairy products), leather industry, and tobacco industry.

Further to the above, the sugars produced from saccharifying the pretreated biomass as described herein may be used to produce in general, organic products, chemicals, fuels, commodity and specialty chemicals such as xylose, acetone, acetate, glycine, lysine, organic acids (e.g., lactic acid), 1,3-propanediol, butanediol, glycerol, ethylene glycol, furfural, polyhydroxyalkanoates, cis, cis-muconic acid, and animal feed (Lynd, L. R., Wyman, C. E., and Gerngross, T. U., Biocommodity Engineering, Biotechnol. Prog., 15: 777-793, 1999; and Philippidis, G. P., Cellulose bioconversion technology, in Handbook on Bioethanol: Production and Utilization, Wyman, C. E., ed., Taylor & Francis, Washington, D.C., 179-212, 1996; and Ryu, D. D. Y., and Mandels, M., Cellulases: biosynthesis and applications, Enz. Microb. Technol., 2: 91-102, 1980).

Potential coproducts may also be produced, such as multiple organic products from fermentable carbohydrate. Lignin-rich residues remaining after pretreatment and fermentation can be converted to lignin-derived chemicals, chemical building blocks or used for power production.

Conventional methods of fermentation and/or saccharification are known in the art including, but not limited to, saccharification, fermentation, separate hydrolysis and fermentation (SHF), simultaneous saccharification and fermentation (SSF), simultaneous saccharification and cofermentation (SSCF), hybrid hydrolysis and fermentation (HHF), and direct microbial conversion (DMC).

SHF uses separate process steps to first enzymatically hydrolyze cellulose to sugars such as glucose and xylose and then ferment the sugars to ethanol. In SSF, the enzymatic hydrolysis of cellulose and the fermentation of glucose to ethanol is combined in one step (Philippidis, G. P., in Handbook on Bioethanol: Production and Utilization, Wyman, C. E., ed., Taylor & Francis, Washington, D.C., 179-212, 1996). SSCF includes the cofermentation of multiple sugars (Sheehan, J., and Himmel, M., Bioethanol, Biotechnol. Prog. 15: 817-827, 1999). HHF includes two separate steps carried out in the same reactor but at different temperatures, i.e., high temperature enzymatic saccharification followed by SSF at a lower temperature that the fermentation strain can tolerate. DMC combines all three processes (cellulase production, cellulose hydrolysis, and fermentation) in one step (Lynd, L. R., Weimer, P. J., van Zyl, W. H., and Pretorius, I. S., Microbiol. Mol. Biol. Reviews, 66: 506-577, 2002).

These processes may be used to produce target products from the readily saccharifiable biomass produced by the pretreatment methods described herein.

### Advantages of the Present Methods:

One of the advantages of the present methods is the high selectivity for removing lignin from the biomass while leaving the carbohydrates largely intact. Less selective pretreatment methods hydrolyze a portion of the carbohydrates to sugars which, being more soluble than cellulose and hemicellulose in the solvent solution, are therefore separated from the carbohydrates in the filtering step. Removal of some of the monomeric sugars with the lignin in the filtering step results in a decrease in the overall yield to sugar (i.e. through a saccharification step). The present methods minimize sugar loss during lignin removal, which is of economic benefit.

Additionally, lignin is more electron rich than the carbohydrates contained in biomass, and as a result the lignin is more prone to oxidation by the Mn(III) salt than are the carbohydrates. While not wishing to be bound by any theory, oxidation of the lignin by the Mn(III) salts is believed to reduce the molecular weight of the lignin fragments, which in turn renders them both more soluble in the solvent solution and less able to bind to cellulolytic enzymes. The present methods advantageously combine the use of organosolv with selective Mn(III)-promoted oxidation of lignin to produce a readily saccharifiable biomass.

Another advantage of the present methods is the use of Mn(III) salts which are readily available and do not require any special syntheses.

### EXAMPLES

The goal of the experimental work described below was to develop a pretreatment process for lignocellulose that maximized lignin extraction and minimized carbohydrate loss in the pretreatment to produce a readily saccharifiable biomass that may be further processed to result in a maximal monomeric sugar yield following enzymatic saccharification. The approach adopted was to selectively fragment and extract the lignin into a suitable solvent in the presence of at least one Mn(III) salt while retaining the sugars with the solids residue. The following experiments show that organosolv treatment in combination with Mn(III)-promoted oxidation selectively extracted and fragmented the lignin from the provided biomass to produce a readily saccharifiable biomass.

The present invention is further defined in the following examples. It should be understood that these examples, while indicating preferred embodiments of the invention, are given by way of illustration only. From the above discussion and these examples, one skilled in the art can ascertain the essential characteristics of this invention, and without departing from the spirit and scope thereof, can make various changes and modifications of the invention to adapt it to various uses and conditions.

### Materials

The following materials were used in the examples. All commercial reagents were used as received.

Sulfuric acid, glucose, xylose, cellobiose, sodium chloride, manganese(III) acetate, and citric acid were obtained from Sigma-Aldrich (St. Louis, Mo.).

Corn cob was purchased from Independence Corn By Products (ICBP Cob), Independence, Iowa. The seller stored the cob at 60° C. and milled and sieved the cob to ⅛″. The dry mass content of the cob was 92.5%.

### Carbohydrate Analysis of Biomass

A modified version of the NREL LAP procedure “Determination of Structural Carbohydrates and Lignin in Biomass” was used to determine the weight percent glucan and xylan in the biomass. Sample preparation was simplified by drying at 80° C. under vacuum or at 105° C. under ambient pressure overnight. The samples were knife milled to pass through a 20 mesh screen but were not sieved. The dry milled solids were than subjected to the acid hydrolysis procedure at a 50 mg solids scale. The solids were not first extracted with water or ethanol. HPLC analysis of sugars was done on an Aminex HPX-87H column and no analysis of lignin was attempted.

The soluble sugars glucose, cellobiose, and xylose in saccharification liquor were measured by HPLC (Agilent 1100, Santa Clara, Calif.) using Bio-Rad HPX-87H column (Bio-Rad Laboratories, Hercules, Calif.) with appropriate guard columns, using 0.01 N aqueous sulfuric acid as the eluant. The sample pH was measured and adjusted to 5-6 with sulfuric acid if necessary. The sample was then passed through a 0.2 um syringe filter directly into an HPLC vial. The HPLC run conditions were as follows:
Biorad Aminex HPX-87H (for carbohydrates):
Injection volume: 10-50 μL, dependent on concentration and detector limits
Mobile phase: 0.01 N aqueous sulfuric acid, 0.2 m filtered and degassed
Flow rate: 0.6 mL/minute
Column temperature: 50° C., guard column temperature <60° C.
Detector temperature: as close to main column temperature as possible
Detector: refractive index
Run time: 15 minute data collection
   After the run, concentrations in the sample were determined from standard curves for each of the compounds.

The following abbreviations are used:

“HPLC” is High Performance Liquid Chromatography, “C” is degrees Centigrade or Celsius; “%” is percent; “w/w” is weight for weight; “mL” is milliliter; “h” is hour(s); “rpm” is revolution per minute; “EtOH” is ethanol; “mg/g” is milligram per gram; “g/100 mL” is gram per 100 milliliter; “g” is gram; “NaOH” is sodium hydroxide; “w/v” is weight per volume; “v/v” is volume for volume, “w/w” is weight for weight; “mm” is millimeter; “mL/min” is milliliter per minute; “min” is minutes; “mM” is millimolar, “N” is normal, “μL” is microliter.

### Example 1

The purpose of this Example was to show the beneficial effect of pretreatment with 100% ethanol in the presence of Mn(OAc)₃ for producing a readily saccharifiable biomass. The beneficial effect was quantified by the glucose and xylose yields obtained upon saccharification of the readily saccharifiable biomass, the pretreated corn cob.

To a slurry of corn cob (2.004 g) in EtOH (8.0 mL) was added Mn(OAc)₃ (0.100 g), and the mixture was heated to 150° C. for six hours in air. Upon cooling, the reaction mixture was filtered and washed with 8 mL ethanol, followed by 8 mL acetone. The residue was dried in vacuo, at room temperature, to afford 1.804 g residue (90% mass recovery) and then ground through a 2 mm sieve. The ground residue, also referred to as pretreated corn cob, was saccharified as follows.

To pretreated corn cob (0.499 g) was added 4.093 mL citrate buffer (pH=5), Accellerase™ 1000 cellulase (46.3 μL, concentration 97.1 mg/mL) and Multifect® CX 12L (26.7 μL, concentration 56.1 mg/mL) enzyme cocktails, and the mixture was left stirring in an incubator/shaker at 48° C. Samples were taken every 24 h and analyzed by HPLC to determine the monomeric sugar yields versus time. Saccharification yields for glucose and xylose are given in Tables 1 and 2.

### Example 2

The purpose of this Example was to show the beneficial effect of pretreatment with 90% ethanol/10% water in the presence of Mn(OAc)₃ for producing a readily saccharifiable biomass. The beneficial effect was quantified by the glucose and xylose yields obtained upon saccharification of the readily saccharifiable biomass, the pretreated corn cob.

To a slurry of corn cob (1.995 g) in a 10% H₂O/90% EtOH mixture (v/v) (8.0 mL) was added Mn(OAc)₃ (0.100 g), and the mixture was heated to 150° C. for six hours in air. Upon cooling, the reaction mixture was filtered and washed with 8 mL ethanol, followed by 8 mL acetone. The residue was dried in vacuo, at room temperature, to afford 1.726 g residue (87% mass recovery) and then ground through a 2 mm sieve. The ground residue, also referred to as pretreated corn cob, was saccharified as follows.

To pretreated corn cob (0.500 g) was added 4.093 mL citrate buffer (pH=5), Accellerase™ 1000 cellulase (46.3 μL, concentration 97.1 mg/mL) and Multifect® CX 12L (26.7 μL, concentration 56.1 mg/mL) enzyme cocktails, and the mixture was left stirring in an incubator/shaker at 48° C. Samples were taken every 24 h and analyzed by HPLC to determine the monomeric sugar yields versus time. Saccharification yields for glucose and xylose are given in Tables 1 and 2.

### Example 3

The purpose of this Example was to show the beneficial effect of pretreatment with 75% ethanol/25% water in the presence of Mn(OAc)₃ for producing a readily saccharifiable biomass. The beneficial effect was quantified by the glucose and xylose yields obtained upon saccharification of the readily saccharifiable biomass, the pretreated corn cob.

To a slurry of corn cob (2.000 g) in a 25% H₂O/75% EtOH mixture (v/v) (8.0 mL) was added Mn(OAc)₃ (0.100 g), and the mixture was heated to 150° C. for six hours in air. Upon cooling, the reaction mixture was filtered and washed with 8 mL ethanol, followed by 8 mL acetone. The residue was dried in vacuo, at room temperature, to afford 1.726 g residue (84% mass recovery) and then ground through a 2 mm sieve. The ground residue, also referred to as pretreated corn cob, was saccharified as follows:

To pretreated corn cob (0.500 g) was added 4.093 mL citrate buffer (pH=5), Accellerase™ 1000 cellulase (46.3 μL, concentration 97.1 mg/mL) and Multifect® CX 12L (26.7 μL, concentration 56.1 mg/mL) enzyme cocktails, and the mixture was left stirring in an incubator/shaker at 48° C. Samples were taken every 24 h and analyzed by HPLC to determine the monomeric sugar yields versus time. Saccharification yields for glucose and xylose are given in Tables 1 and 2.

### Example 4

The purpose of this Example was to show the beneficial effect of pretreatment with 50% ethanol/50% water in the presence of Mn(OAc)₃ for producing a readily saccharifiable biomass. The beneficial effect was quantified by the glucose and xylose yields obtained upon saccharification of the readily saccharifiable biomass, the pretreated corn cob.

To a slurry of corn cob (2.000 g) in a 50% H₂O/50% EtOH mixture (v/v) (8.0 mL) was added Mn(OAc)₃ (0.100 g), and the mixture was heated to 150° C. for six hours in air. Upon cooling, the reaction mixture was filtered and washed with 8 mL ethanol, followed by 8 mL acetone. The residue was dried in vacuo, at room temperature, to afford 1.726 g residue (78% mass recovery) and then ground through a 2 mm sieve. The ground residue, also referred to as pretreated corn cob, was saccharified as follows.

To pretreated corn cob (0.500 g) was added 4.093 mL citrate buffer (pH=5), Accellerase™ 1000 cellulase (46.3 μL, concentration 97.1 mg/mL) and Multifect® CX 12L (26.7 μL, concentration 56.1 mg/mL) enzyme cocktails, and the mixture was left stirring in an incubator/shaker at 48° C. Samples were taken every 24 h and analyzed by HPLC to determine the monomeric sugar yields versus time. Saccharification yields for glucose and xylose are given in Tables 1 and 2.

### Example 5

The purpose of this Example was to show the beneficial effect of pretreatment with 25% ethanol/75% water in the presence of Mn(OAc)₃ for producing a readily saccharifiable biomass. The beneficial effect was quantified by the glucose and xylose yields obtained upon saccharification of the readily saccharifiable biomass, the pretreated corn cob.

To a slurry of corn cob (1.998 g) in a 75% H₂O/25% EtOH mixture (v/v) (8.0 mL) was added Mn(OAc)₃ (0.100 g), and the mixture was heated to 150° C. for six hours in air. Upon cooling, the reaction mixture was filtered and washed with 8 mL ethanol, followed by 8 mL acetone. The residue was dried in vacuo, at room temperature, to afford 1.726 g residue (73% mass recovery) and then ground through a 2 mm sieve. The ground residue, also referred to as pretreated corn cob, was saccharified as follows.

To pretreated corn cob (0.500 g) was added 4.093 mL citrate buffer (pH=5), Accellerase™ 1000 cellulase (46.3 μL, concentration 97.1 mg/mL) and Multifect® CX 12L (26.7 μL, concentration 56.1 mg/mL) enzyme cocktails, and the mixture was left stirring in an incubator/shaker at 48° C. Samples were taken every 24 h and analyzed by HPLC to determine the monomeric sugar yields versus time. Saccharification yields for glucose and xylose are given in Tables 1 and 2.

### Example 6

The purpose of this Example was to show the beneficial effect of pretreatment with 10% ethanol/90% water in the presence of Mn(OAc)₃ for producing a readily saccharifiable biomass. The beneficial effect was quantified by the glucose and xylose yields obtained upon saccharification of the readily saccharifiable biomass, the pretreated corn cob.

To a slurry of corn cob (2.005 g) in a 90% H₂O/10% EtOH mixture (v/v) (8.0 mL) was added Mn(OAc)₃ (0.095 g), and the mixture was heated to 150° C. for six hours in air. Upon cooling, the reaction mixture was filtered and washed with 8 mL ethanol, followed by 8 mL acetone. The residue was dried in vacuo, at room temperature, to afford 1.726 g residue (71% mass recovery) and then ground through a 2 mm sieve. The ground residue, also referred to as pretreated corn cob, was saccharified as follows.

To pretreated corn cob (0.500 g) was added 4.093 mL citrate buffer (pH=5), Accellerase™ 1000 cellulase (46.3 μL, concentration 97.1 mg/mL) and Multifect® CX 12L (26.7 μL, concentration 56.1 mg/mL) enzyme cocktails, and the mixture was left stirring in an incubator/shaker at 48° C. Samples were taken every 24 h and analyzed by HPLC to determine the monomeric sugar yields versus time. Saccharification yields for glucose and xylose are given in Tables 1 and 2.

### Example 7

The purpose of this Example was to show the beneficial effect of pretreatment with 100% water in the presence of Mn(OAc)₃ for producing a readily saccharifiable biomass. The beneficial effect was quantified by the glucose and xylose yields obtained upon saccharification of the readily saccharifiable biomass, the pretreated corn cob.

To a slurry of corn cob (1.996 g) in H₂O (8.0 mL) was added Mn(OAc)₃ (0.095 g), and the mixture was heated to 150° C. for six hours in air. Upon cooling, the reaction mixture was filtered and washed with 8 mL ethanol, followed by 8 mL acetone. The residue was dried in vacuo, at room temperature, to afford 1.350 residue (67% mass recovery) and then ground through a 2 mm sieve. The ground residue, also referred to as pretreated corn cob, was saccharified as follows.

To pretreated corn cob (0.501 g) was added 4.093 mL citrate buffer (pH=5), Accellerase™ 1000 cellulase (46.3 μL, concentration 97.1 mg/mL) and Multifect® CX 12L (26.7 μL, concentration 56.1 mg/mL) enzyme cocktails, and the mixture was left stirring in an incubator/shaker at 48° C. Samples were taken every 24 h, and analyzed by HPLC, to determine the monomeric sugar yields versus time. Saccharification yields for glucose and xylose are given in Tables 1 and 2.

TABLE 1
Saccharification percent yields of glucose.

| | Example | | | | | | |
|---|---|---|---|---|---|---|---|
| Sacch. Time | 1 | 2 | 3 | 4 | 5 | 6 | 7 |
| 6 h | 10.7 | 19.4 | 18.5 | 15.0 | 13.6 | 11.9 | 9.6 |
| 24 h | 35.1 | 45.4 | 48.4 | 53.6 | 47.9 | 43.9 | 31.4 |
| 48 h | 38.2 | 50.5 | 57.5 | 56.6 | 49.8 | 45.9 | 45.7 |

TABLE 2
Saccharification percent yields of xylose.

| | Example | | | | | | |
|---|---|---|---|---|---|---|---|
| Sacch. Time | 1 | 2 | 3 | 4 | 5 | 6 | 7 |
| 6 h | 22.5 | 16.8 | 17.7 | 19.8 | 20.5 | 17.5 | 12.5 |
| 24 h | 31.8 | 40.5 | 44.8 | 53.3 | 42.1 | 38.2 | 29.5 |
| 48 h | 33.5 | 45.3 | 57.6 | 64.2 | 50.6 | 46.4 | 37.0 |

The results show that ethanol/water treatment solutions containing about 0 percent to about 100 percent (v/v) ethanol, when used in the presence of at least one Mn(III) salt, were effective in producing a readily saccharifiable biomass, as demonstrated by the yields of glucose and xylose after saccharification of the biomass. The highest sugar yields, reflecting the most readily saccharifiable biomass samples, were obtained with the most effective solvent/water treatment solutions, which contained about 25 percent to about 75 percent (v/v) ethanol, in the presence of at least one Mn(III) salt. Solvent/water treatment solutions containing about 10 percent to about 90 percent (v/v) ethanol were more effective than 100% ethanol solutions and, in most cases, more effective than 100% water, in the presence of at least one Mn(III) salt.

The following Comparative Examples were performed without Mn(III).

### Comparative Example A

Comparative Example A was performed in the same way as Example 1, but without Mn(OAc)₃. The purpose of this Comparative Example was to show the effect of pretreatment with 100% ethanol in the absence of Mn(OAc)₃. The effect was quantified by the glucose and xylose yields obtained upon saccharification of the pretreated corn cob.

A slurry of corn cob (2.002 g) in EtOH (8.0 mL) was heated to 150° C. for six hours in air. Upon cooling, the reaction mixture was filtered and washed with 8 mL ethanol, followed by 8 mL acetone. The residue was dried in vacuo, at room temperature, to afford 1.755 g residue (83.3% mass recovery). The residue was then ground through a 2 mm sieve and saccharified as follows.

To the residue (0.499 g) was added 2.994 mL citrate buffer (pH=5), Accellerase™ 1000 cellulase (46.3 μL, concentration 97.1 mg/mL) and Multifect® CX 12L (26.7 μL, concentration 56.1 mg/mL) enzyme cocktails, and the mixture was left stirring in an incubator/shaker at 48° C. Samples were taken every 24 h, and analyzed by HPLC, to determine the monomeric sugar yields versus time. Saccharification yields for glucose and xylose are given in Tables 3 and 4.

### Comparative Example B

Comparative Example B was performed in the same way as Example 4, but without Mn(OAc)₃. The purpose of this Comparative Example was to show the effect of pretreatment with 50% H₂O/50% EtOH mixture (v/v) in the absence of Mn(OAc)₃. The effect was quantified by the glucose and xylose yields obtained upon saccharification of the pretreated corn cob.

A slurry of corn cob (2.000 g) in a 50% H₂O/50% EtOH mixture (v/v) (8.0 mL) was heated to 150° C. for six hours in air. Upon cooling, the reaction mixture was filtered and washed with 8 mL ethanol, followed by 8 mL acetone. The residue was dried in vacuo, at room temperature, to afford 1.602 g residue (76.1% mass recovery). The residue was then ground through a 2 mm sieve and saccharified as follows.

To the residue (0.499 g) was added 2.994 mL citrate buffer (pH=5), Accellerase™ 1000 cellulase (46.3 μL, concentration 97.1 mg/mL) and Multifect® CX 12L (26.7 μL, concentration 56.1 mg/mL) enzyme cocktails, and the mixture was left stirring in an incubator/shaker at 48° C. Samples were taken every 24 h, and analyzed by HPLC, to determine the monomeric sugar yields versus time. Saccharification yields for glucose and xylose are given in Tables 3 and 4.

### Comparative Example C

Comparative Example C was performed in the same way as Example 7, but without Mn(OAc)₃. The purpose of this Comparative Example was to show the effect of pretreatment with 100% water in the absence of Mn(OAc)₃. The effect was quantified by the glucose and xylose yields obtained upon saccharification of the pretreated corn cob.

A slurry of corn cob (2.002 g) in H₂O (8.0 mL) was heated to 150° C. for six hours in air. Upon cooling, the reaction mixture was filtered and washed with 8 mL ethanol, followed by 8 mL acetone. The residue was dried in vacuo, at room temperature, to afford 1.363 g residue (64.7% mass recovery). The residue was then ground through a 2 mm sieve and saccharified as follows.

To the residue (0.499 g) was added 2.994 mL citrate buffer (pH=5), Accellerase™ 1000 cellulase (46.3 μL, concentration 97.1 mg/mL) and Multifect® CX 12L (26.7 μL, concentration 56.1 mg/mL) enzyme cocktails, and the mixture was left stirring in an incubator/shaker at 48° C. Samples were taken every 24 h, and analyzed by HPLC, to determine the monomeric sugar yields versus time. Saccharification yields for glucose and xylose are given in Tables 3 and 4.

TABLE 3
Saccharification percent yields of glucose for the Comparative
Examples.

| Saccharification | Comparative Example | | |
|---|---|---|---|
| Time | A | B | C |
| 24 h | 18.6 | 24.6 | 19.2 |
| 48 h | 27.4 | 35.0 | 28.3 |

TABLE 4
Saccharification percent yields of xylose for the Comparative
Examples.

| Saccharification | Comparative Example | | |
|---|---|---|---|
| Time | A | B | C |
| 24 h | 12.7 | 22.5 | 13.9 |
| 48 h | 18.6 | 28.2 | 17.9 |

Comparison of the glucose and xylose yields of the Comparative Examples with those of the inventive Examples shows that substantially higher yields were obtained under the same reaction conditions in the presence of at least one Mn(III) salt. The higher sugar yields demonstrate that the present method comprising contacting biomass with a solvent/water treatment solution in the presence of at least one Mn(III) salt according to the method produces a readily saccharifiable biomass.

Although particular embodiments of the present invention have been described in the foregoing description, it will be understood by those skilled in the art that the invention is capable of numerous modifications, substitutions, and rearrangements without departing from the spirit of essential attributes of the invention. Reference should be made to the appended claims, rather than to the foregoing specification, as indicating the scope of the invention.

What is claimed is:

1. A method for producing readily saccharifiable carbohydrate-enriched biomass, the method comprising:
(a) providing lignocellulosic biomass comprising lignin;
(b) contacting the biomass with a solvent solution in the presence of at least one Mn(III) salt whereby a biomass-solvent suspension is formed;
(c) heating the biomass-solvent suspension to a temperature of about 100° C. to about 220° C. for a reaction time of about 15 minutes to about 48 hours whereby lignin is fragmented from the biomass and said lignin is dissolved in the suspension; and
(d) filtering an amount of free liquid under pressure after heating the suspension in (c) whereby the dissolved lignin is removed and whereby readily saccharifiable carbohydrate-enriched biomass is produced.

2. The method of claim 1, further comprising:
(e) washing the readily saccharifiable biomass produced in step (d) with the solvent solution.

3. The method of claim 2, further comprising repeating steps (b)-(e) one or more times.

4. The method of claim 1, wherein the lignocellulosic biomass is subjected to preprocessing prior to step (a).

5. The method of claim 1, wherein the Mn(III) salt is selected from the group consisting of manganese(III) triacetate, manganese(III) acetylacetonate, and combinations thereof.

6. The method of claim 1, wherein the Mn(III) salt is at a concentration of up to about 15% by weight of dry biomass.

7. The method of claim 6, wherein the concentration is up to about 10%.

8. The method of claim 7, wherein the concentration is up to about 5%.

9. The method of claim 1, wherein the temperature is about 140° C. to about 180° C.

10. The method of claim 1, wherein the reaction time is about 1 hour to about 12 hours.

11. The method of claim 1, wherein the solvent solution comprises an alcohol selected from the group consisting of methanol, ethanol, n-propanol, isopropanol, n-butanol, 2-butanol, isobutanol, and t-butanol, and mixtures of these.

12. The method of claim 11, wherein the alcohol is ethanol.

13. The method of claim 12, wherein the solvent solution contains about 0 to about 100 percent (volume/volume) ethanol.

14. The method of claim 13, wherein the solvent solution contains about 10 percent to about 90 percent (volume/volume) ethanol.

15. The method of claim 14, wherein the solvent solution contains about 25 percent to about 75 percent (volume/volume) ethanol.

16. The method of claim 1, wherein the dry weight of biomass is at a concentration of from about 15% to about 70% of the weight of the biomass-solvent suspension.

17. The method of claim 16, wherein the dry weight of biomass is from about 20% to about 50%.

18. The method of claim 1, wherein the heated suspension of step (c) is cooled to room temperature before filtering in step (d).

19. The method of claim 1, further comprising drying the filtered biomass after step (d).

20. The method of claim 1, further comprising saccharifying the readily saccharifiable biomass with an enzyme consortium whereby fermentable sugars are produced.

21. The method of claim 20, further comprising fermenting the sugars to produce a target product.

22. The method of claim 21, wherein the target product is selected from the group consisting of ethanol, butanol, and 1,3-propanediol.

23. A method for selectively removing lignin from biomass, the method comprising:
(a) providing lignocellulosic biomass having a carbohydrate content and comprising lignin;
(b) contacting the biomass with a solvent solution comprising water in the presence of at least one Mn(III) salt whereby a biomass-solvent suspension is formed;
(c) heating the biomass-solvent suspension to a temperature of about 100° C. to about 220° C. for a reaction time of about 15 minutes to about 48 hours whereby lignin is fragmented from the biomass and is dissolved in the suspension; and
(d) filtering free liquid under pressure after heating the suspension in (c) whereby dissolved lignin is removed and wherein the carbohydrate content of the biomass is highly conserved.

24. The method of claim 23, wherein the Mn(III) salt is selected from the group consisting of manganese(III) triacetate, manganese(III) acetylacetonate, and combinations thereof.

25. The method of claim 1 or 23 wherein said Mn(III) salt of step (b) is formed in situ by oxidation of a Mn(II) salt or by reaction of a Mn(III) compound.

26. The method of claim 25, wherein the Mn(III) salt is formed by reaction of an Mn(II) compound selected from the group consisting of acetate and acetylacetonate groups.
